(19) United States
(12) Patent Application Publication
(10) Pub. No.: US 2026/0337304 A1
(43) Pub. Date: Oct. 29, 2026

(54) PROCESS FOR CONJUGATION OF HYALURONIC ACID AND CONJUGATES OF HYALURONIC ACID SO OBTAINED

(71) Applicant: GLYCOCORE PHARMA S.R.L., Como (IT)

(72) Inventors: Giulio Bianchini, COMO (IT); Emiliano Esposito, COMO (IT)

(73) Assignee: GLYCOCORE PHARMA S.R.L., Como (IT)

(21) Appl. No.: 19/108,940

(22) PCT Filed: Sep. 8, 2023
(86) PCT No.: PCT/IB2023/058903

§ 371 Date: Mar. 5, 2025

(30) Foreign Application Priority Data
Sep. 12, 2022 (IT) 102022000018585

(51) Int. Cl. C08B 37/08; A61K 47/36; B01D 15/36; B01D 61/24; C08B 37/00

(52) U.S. Cl. CPC C08B 37/0072; A61K 47/36; B01D 15/361; B01D 61/243; C08B 37/0003

(57) ABSTRACT

The present invention describes a process for conjugating hyaluronic acid by using a specific condensing agent and under certain operating conditions which allow to obtain derivatives of hyaluronic acid with disaccharides or oligosaccharides having the desired degree of conjugation in an efficient, fast, and economical way. Hyaluronic acid conjugates having specific degrees of conjugation are also described, and likewise the uses thereof for therapeutic purposes and as scaffolds for biomaterials.

### FIELD OF THE INVENTION

The present invention relates to a process for conjugating hyaluronic acid by using a specific condensing agent and under certain operating conditions which allow to obtain derivatives of hyaluronic acid with disaccharides or oligosaccharides having the desired degree of conjugation in an efficient, fast, and economical way.

The present invention therefore also relates to hyaluronic acid conjugates having specific degrees of conjugation and uses thereof.

### BACKGROUND ART

Hyaluronic acid, or ‘HA’ for brevity, is a non-sulfated linear glycosaminoglycan (GAG) consisting of repetitive disaccharide units consisting of a glucuronic acid residue and an N-Acetylglucosamine residue, linked together alternatively by β1→4 and β1→3 glycosidic bonds.

At physiological pH, the carboxyl groups of the glucuronic units are ionised, giving HA high polarity and consequently good solubility in water. The molecular weight of HA can vary between 10³ and 10⁷ Da, and this parameter affects the properties thereof, thus making different applications possible.

HA is present naturally in both vertebrate organisms and bacteria. Its presence is mainly in the extracellular matrix (ECM) of epithelial cells and connective tissues. The biological properties of hyaluronic acid are attributable to the chemical and physical characteristics thereof, such as viscosity and high water retention capacity, which allow said hyaluronic acid to perform a structural and hydrating role. Today, studies of hyaluronic acid concern its role as a drug carrier through appropriate chemical functionalisations. The cellular internalisation of hyaluronic acid by means of CD44 receptors during enzymatic degradation of said hyaluronic acid determines the intracellular release of the drug obtained through conjugation with HA or entrapment in HA particles. Along this line, the possibility of functionalising hyaluronic acid with modified lactose is particularly interesting given the biological significance in terms of interaction with galectins.

Indeed, galectins are galactose-specific receptors, so the addition of this residue to the HA chain allows the polymer to interact not only with the CD44 receptors but also with galectins, producing a desired synergistic effect.

Numerous derivatives of hyaluronic acid are known in the literature, for the most varied applications. However, it has also been observed that there is still a need for a functionalisation process that is efficient, economical, and above all capable of guaranteeing achievement of the desired degree of substitution and a high yield, while also minimising by-products.

### SUMMARY OF THE INVENTION

Said object was achieved by a process for conjugating hyaluronic acid through a specific condensing agent and under certain operating conditions, as stated in claim 1.

In another aspect, the present invention concerns a conjugate of hyaluronic acid, or a pharmaceutically acceptable salt thereof, and a disaccharide or an oligosaccharide having an average degree of substitution in the carboxyl group of 10-50%, more preferably 15-45%.

In a further aspect, the present invention relates to the use of said conjugate in the treatment of pathologies ascribable to altered galectin expression. Non-limiting examples of such pathologies are non-alcoholic steatohepatitis, plaque psoriasis, rheumatoid arthritis, osteoarthritis, neoplasia, adhesions, and fibrotic dermal, pulmonary, renal, and cardiovascular processes.

In a still further aspect, the present invention relates to the use of such conjugate in rheumatology, orthopaedics, oncology, plastic/cosmetic surgery, haemodialysis, cardiology, angiology, ophthalmology, otolaryngology, pneumology, odontology, gynaecology, urology, dermatology, oncology, and tissue repair.

The characteristics and advantages of the present invention will become apparent from the following detailed description and the embodiments provided as illustrative, non-limiting examples.

### DETAILED DESCRIPTION OF THE INVENTION

The invention therefore relates to a process for conjugating hyaluronic acid, or a pharmaceutically acceptable salt thereof, with a disaccharide or an oligosaccharide, comprising the following steps:
i) providing an aqueous solution of hyaluronic acid or a pharmaceutically acceptable salt thereof,
ii) adding a triazine compound of formula (I), under stirring, to form an ester adduct between the carboxyl group of hyaluronic acid, or a pharmaceutically acceptable salt thereof, and the triazine ring of the compound of formula (I),
iii) waiting at least 15 minutes and adding a primary amine of a disaccharide or oligosaccharide, under stirring, to form a conjugate of hyaluronic acid, or a pharmaceutically acceptable salt thereof, with said disaccharide or oligosaccharide, via amide bond,
iv) promoting precipitation of the conjugate thus obtained, adding a water-miscible organic solvent, thus obtaining a crude conjugate, and possibly washing the precipitated conjugate with further organic solvent possibly mixed with water,
v) separating the crude conjugate, and optionally
vi) purifying the crude conjugate by reprecipitation, by ion-exchange resin treatment, by dialysis, or by the combination and/or repetition thereof, and
vii) obtaining the purified conjugate by drying,
   the process being carried out at a temperature of 4-100 °C and at a pH of 5-8,
   wherein said triazine compound of formula (I) is:

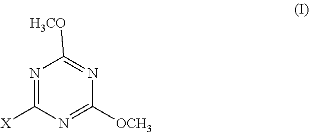

wherein X is a halogen chosen from I, Br, Cl, and mixtures thereof, or is a group —NR₁R₂ selected from NH₂, NMe₂, NHPr, PhNH, 4-MeC₆H₄NH,

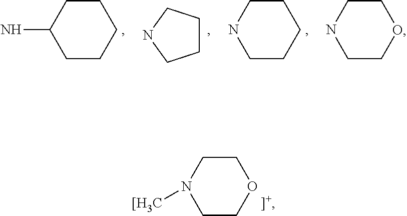

and mixtures thereof.

As can also be seen from the following examples, the process according to the invention allows the conjugate to be obtained, conveniently and quickly, with the desired degree of substitution and likewise with high purity.

In step i), an aqueous solution of hyaluronic acid or a pharmaceutically acceptable salt thereof is provided.

Preferably, said hyaluronic acid in step i) has an average molecular weight of 50-5000 kDa, more preferably 50-500 kDa.

The term “pharmaceutically acceptable salt” of hyaluronic acid in step i) means a salt preferably selected from sodium hyaluronate, potassium hyaluronate, calcium hyaluronate, magnesium hyaluronate, zinc hyaluronate, cobalt hyaluronate, ammonium hyaluronate, tetrabutylammonium hyaluronate, and mixtures thereof.

Preferably, step i) takes place at a pH of 5-7, more preferably 6-7, upon addition of an acid, preferably 2-[N-morpholino]-ethanesulfonic acid (MES).

In step ii), a triazine compound of formula (I) is added, under stirring, to form an ester adduct between the carboxyl group of hyaluronic acid, or a pharmaceutically acceptable salt thereof, and the triazine ring, as shown below:

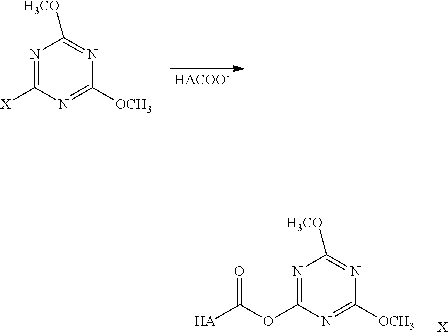

where “HACOO” is the carboxyl group of hyaluronic acid, or a pharmaceutically acceptable salt thereof, which binds to the triazine ring and forms the ester adduct on the right, releasing group X.

Preferably, said triazine compound of formula (I) is 4,6-Dimethoxy-1,3,5-triazin-2-yl)-4-methylmorpholinium chloride, (‘DMTMM’ for brevity), 2-Chloro-4,6-dimethoxy-1,3,5-triazine (‘CDMT’ for brevity), or a mixture thereof.

In particularly preferred embodiments, said triazine compound of formula (I) is DMTMM, and in step ii), the following reaction occurs:

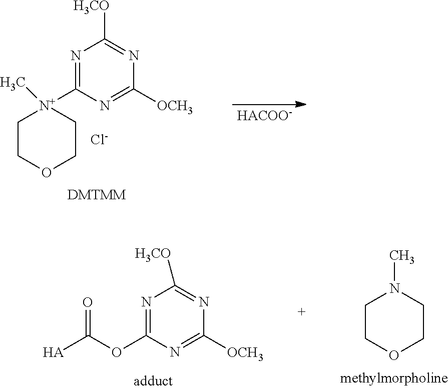

where “HACOO” is the carboxyl group of hyaluronic acid, or a pharmaceutically acceptable salt thereof, which binds to the triazine ring of DMTMM and forms the ester adduct on the right, releasing methylmorpholine.

Preferably, the hyaluronic acid or pharmaceutically acceptable salt thereof and the triazine compound of formula (I), in step ii), are in a molar ratio of 3:1 to 1:3.

The mixture that forms after the addition of the triazine compound of formula (I), is left to react for at least 15 minutes, preferably at least 30 minutes, after which the primary amine of a disaccharide or an oligosaccharide is added, under stirring, to form a conjugate of hyaluronic acid, or a pharmaceutically acceptable salt thereof, with said disaccharide or oligosaccharide, via amide bond, as per step iii).

The term “primary amine” refers not only to the free primary amine —NH₂, but also to pharmaceutically acceptable salts thereof.

Preferably, the term “oligosaccharide” means a saccharide polymer comprising 3 to 6 monosaccharides, i.e. from a trisaccharide to hexasaccharide.

More preferably, said primary amine of a disaccharide or an oligosaccharide is a primary amine derivative of sucrose, maltose, lactose, trehalose, gentiobiose, cellobiose, maltotriose, raffinose, stachyose, maltotetraose, melezitose, isomaltose, panose, nystose, 1-ketose, galactopinitol, galactosyllactose, or a mixture thereof.

In preferred embodiments, in step iii), a primary amine of a disaccharide is added which is a primary amine derivative of sucrose, maltose, lactose, trehalose, gentiobiose, cellobiose or a mixture thereof, more preferably a primary amine derivative of lactose. The addition of said primary amine to the mixture containing the ester adduct described above leads to the formation of a conjugate, in addition to the release of 4,6-dimethoxy-1,3,5-triazin-2-ol, as illustrated below:

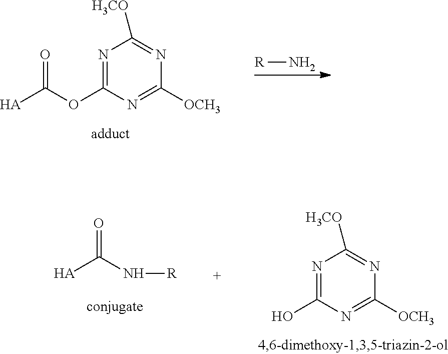

where R—NH₂ is the primary amine of a disaccharide or an oligosaccharide, denoted “R” for brevity, while the resulting conjugate is schematically represented highlighting the amide bond between the carboxyl group of hyaluronic acid, or pharmaceutically acceptable salt thereof, and the amino group of the primary amine.

Preferably, the hyaluronic acid or pharmaceutically acceptable salt thereof and said primary amine, in step iii), are in a molar ratio of 3:1 to 1:3.

Preferably in step iii), the primary amine and the adduct are left to react at room temperature for at least 12 hours, preferably at least 24 hours, more preferably at least 48 hours.

In preferred embodiments, in step iii), after the at least 12 hours left for the reaction, a base is subsequently added, preferably NaOH 5M. This basification step breaks the ester bonds that form—through secondary reactions of the activated carboxylic acid of HA—with the hydroxyl groups thereof or with lactosamine.

In step iv), the precipitation of the conjugate thus obtained is promoted by adding an acid, preferably HCl 5M, and a water-miscible organic solvent, and obtaining a crude conjugate and possibly washing the precipitated conjugate with further organic solvent possibly mixed with water.

The organic solvent serves to reduce the polarity of the water in order to increase the conjugate precipitation speed and yield.

Preferably, said water-miscible organic solvent is acetone, acetonitrile, 1-4 dioxane, tetrahydrofuran or an alcohol, preferably with a number of carbon atoms of 1 to 5, more preferably selected from methanol, ethanol, 2-propanol, or a mixture thereof.

In step v), the crude conjugate is separated from the solvent mixture. Preferably, this separation can be carried out by techniques such as decantation, filtration, evaporation, or centrifugation, or a combination thereof.

In the optional step vi), the crude conjugate thus separated undergoes a further purification procedure, which can be carried out by reprecipitation, ion-exchange resin treatment, or dialysis, to obtain a more purified conjugate. The three purification methods can also be carried out in reciprocal combination and can be repeated several times, e.g. reprecipitation, followed by ion-exchange resin treatment, followed by reprecipitation.

Preferably, when the purification of the crude conjugate in step vi) is by ion-exchange resin treatment, the following sub-steps are carried out:
vi-a) dissolving the crude conjugate in water to obtain an aqueous solution,
vi-b) ion-exchange resin treating said aqueous solution, by using water as an eluting agent,
vi-c) isolating the aqueous solution from the ion exchange resin,
vi-d) bringing the isolated solution to pH neutral.

In preferred embodiments, said cation exchange column is an Amberlite™ IR120 (CAS n.78922-04-0; IUPAC name: 3-[(3-chlorophenyl)sulfonylamino]benzoic acid).

In sub-step vi-d), the eluate can be brought to neutral pH by adding a salt such as NaHCO₃. Subsequent drying, meanwhile, can be carried out by freeze-drying.

The unreacted primary amine retained in the cation exchange column can subsequently be recovered using NH₃ as an eluting agent.

Alternatively, when the crude conjugate purification in step vi) is performed by dialysis, the following sub-steps are carried out:
vi-i) dissolving the crude conjugate in water to obtain an aqueous solution,
vi-ii) dialysing the solution obtained against aqueous saline solution and/or water through an appropriate membrane.

Alternatively and preferably, when the crude conjugate purification in step vi) is performed by reprecipitation, the following sub-steps are carried out:
vi-α) dissolving the crude conjugate in water to obtain an aqueous solution,
vi-β) adding an water-miscible organic solvent, which can the same as or different from the step iv) solvent, thereby promoting reprecipitation of the conjugate,
vi-γ) washing the precipitated conjugate with further organic solvent, thereby obtaining the purified conjugate.

In sub-step vi-β), a saline solution is preferably furthermore added, more preferably a 0.5 M NaCl solution.

In sub-step vi-γ), the action of washing the conjugate with further organic solvent can be repeated several times in order to increase the final purity of said conjugate.

Alternatively and preferably, when the crude conjugate purification in step vi) is performed by dialysis, the following sub-steps are carried out:
vi-●) dissolving the crude conjugate in water to obtain an aqueous solution,
vi-●●) transferring to a 6-8 kDa membrane and dialysing for at least 24 hours against an NaCl saline solution,
vi-●●●) collecting and freeze-drying the retentate.

In preferred embodiments, the process according to the invention is conducted at a temperature of 15-75 °C and a pH of 6-11.

The primary amines described above, and likewise the related pharmaceutically acceptable salts, are commercially available, or alternatively they can be prepared with commonly known methods of amination of disaccharides or oligosaccharides or even by a process comprising the following steps:
reacting said disaccharide or oligosaccharide with ammonium acetate, in the presence of ammonia and NaCNBH₃ under stirring and at a temperature of at least 60 °C,
drying the product thus obtained and placing in a cation exchange column, eluting with deionised water first, to desalinate the product, and then with ammonia,
collecting the eluate that comes out of the column, bringing said eluate to neutral pH, and then drying to obtain the purified primary amine.

Furthermore, said primary amines can be prepared by a process comprising the following steps:
reacting said disaccharide or oligosaccharide with benzylamine, in the presence of a borane under stirring and at a temperature of between 5-60 °C,
isolating the benzylamine derivative obtained from the reductive amination reaction,
reacting said benzylamine derivative with a source of hydrogen in the presence of a catalyst for the hydrogenolysis reaction under stirring and at a temperature of between 4-100 °C,
isolating the benzylamine derivative and purifying the latter through passage through an ion exchange column to desalinate and then dry the product.

The term “borane” includes sodium borohydride, sodium cyanoborohydride, sodium acetate borohydride, sodium triacetoxyborohydride, lithium borohydride, potassium borohydride, tetrabutylammonium borohydride, calcium borohydride, magnesium borohydride, tetraethylammonium borohydride, methyltrioctylammonium borohydride, bis(triphenylphosphine)copper(I)borohydrate, potassium tri(1-pyrazolyl) borohydride, cetyltrimethylammonium borohydride, borane tetrahydrofuran complex, picoline-borane complex, 5-ethyl-2-methylpyridine-borane complex, dimethylsulfide-borane complex, pyridine-borane complex, trimethylamine-borane complex, triethylamine-borane complex, morpholine-borane complex, t-butylamine-borane complex, ammonia-borane complex, diphenylphosphine-borane complex, 4-methylmorpholine-borane complex, borane ethylenediamine complex, or a mixture thereof.

The term “catalyst” preferably refers to Raney catalyst of metal groups 8-11, carbon-supported metal groups 8-11, silica, alumina, aluminosilicates, zirconia, or a mixture thereof.

The term “hydrogen source” refers to H₂ or a compound capable of releasing H+ ions under the conditions of the process.

In a further aspect, the present invention concerns a conjugate of hyaluronic acid, or a pharmaceutically acceptable salt thereof, and a disaccharide or an oligosaccharide having an average degree of substitution of the carboxyl group of 10-50%, more preferably 15-45% with the amide bond to said disaccharide or oligosaccharide, which can be obtained from the process described above, in particular when:
the hyaluronic acid or pharmaceutically acceptable salt thereof and the triazine compound of formula (I), in step ii), are in a molar ratio of 3:1 to 1:3, and/or
the hyaluronic acid or pharmaceutically acceptable salt thereof and said primary amine, in step iii), are in a molar ratio of 3:1 to 1:3.

Indeed, it has been surprisingly observed that by modulating the ratios between the reagents as stated above, it is possible to obtain the conjugate with the desired degree of substitution in an effective and quantitative manner.

Said conjugate can be administered by inhalation, oral, nasal, ocular, urogynaecological, intra-articular, intramuscular, intravenous, intradermal, transdermal, subcutaneous, or external or internal topical route, for example by surgical route.

Preferably, the conjugate is administered by oral, nasal, ocular, urogynaecological, intra-articular, intradermal, or inhalation route.

In certain embodiments, the conjugate is in an injectable form suitable for hard or soft body tissues, such as organs, adipose tissues, mucous membranes, and gums, preferably by intradermal, subcutaneous, intramuscular, or intra-articular route.

The conjugate can be in the form of soft gel capsules or in solid form, such as a tablet, a mini-tablet, a micro-tablet, a granule, a micro-granule, pellet, multiparticulate or micronised particulate, or powder or in the form of a solution, emulsion, gel, ointment, drops, nebuliser solution, or spray.

In preferred embodiments, the conjugate is in the form of a powder, solution, emulsion, gel, ointment, drops, nebuliser solution, spray, or injectable form, for administration by nasal or inhalation or intra-articular or intradermal or ophthalmic or urogynaecological route.

In a still further aspect, the present invention relates to said conjugate in the treatment of pathologies ascribable to altered galectin expression. Non-limiting examples of such pathologies are non-alcoholic steatohepatitis, plaque psoriasis, rheumatoid arthritis, osteoarthritis, neoplasia, adhesions, and fibrotic dermal, pulmonary, renal, and cardiovascular processes. Examples of neoplasia and fibrotic processes include acute lymphoblastic leukaemia, idiopathic pulmonary fibrosis, hepatic fibrosis, cardiac fibrosis, renal fibrosis, and ovarian, prostate, lung, stomach, skin, thyroid, and pancreas tumours.

In a further aspect, the present invention concerns the use of said conjugate as a biomaterial or a scaffold for cell growth, in the treatment of orthopaedic diseases.

In further preferred aspects, the present invention concerns the use of said conjugate in tissue repair or reconstruction, preferably in the creation or replacement of biological tissues or in filling biological tissues, such as filling skin, depressions, bone cartilage, or joints.

In a still further aspect, the present invention concerns the use of said conjugate in rheumatology, orthopaedics, oncology, plastic/cosmetic surgery, haemodialysis, cardiology, angiology, ophthalmology, otolaryngology, pneumology, odontology, gynaecology, urology, dermatology, oncology, and tissue repair; furthermore, the present invention concerns the use of said conjugate in traumatic and/or post-surgical tissue processes and/or chronic fibrotic processes associated with autoimmune diseases, in traumatic and post-surgical sequelae involving the dermis and abdominal tissues, or in post-surgical sequelae of endonasal procedures, in post-surgical sequelae of tendon and/or cartilaginous tissues.

Particularly preferred is the use of the conjugate according to the invention in the treatment of asthma, COPD, IPF, tonsillitis, laryngitis, pharyngitis, nasopharyngitis, sinusitis, rhinitis, tracheitis, hoarseness, and inflammation of the vocal cords with or without dysphonia.

The conjugate can also be used in the cosmetic and dermatological field, in dermatological or cosmetic products, or as a biomedical product, preferably as a bio-absorbable implant.

In a still further aspect, the present invention relates to the use of such conjugate in psoriasis and psoriatic osteoarthritis.

In another aspect, the present invention concerns a composition comprising the conjugate described above and at least one pharmacologically active substance and/or at least one substance with an optionally biological function.

Suitable pharmacologically active substances include: antibiotics, anti-infectives, antimicrobials, antivirals, cytostatics, cytotoxics, anti-tumour drugs, anti-inflammatory drugs, cicatrizants, anaesthetics, analgesics, vasoconstrictors, cholinergic or adrenergic agonists and antagonists, antithrombotics, anticoagulants, haemostatics, fibrinolytics, thrombolytics, proteins and fragments thereof, peptides, polynucleotides, growth factors, enzymes, vaccines, or combinations thereof.

Preferably, said substance with an optionally biological function is chosen from collagen, fibrinogen, fibrin, alginic acid, sodium alginate, potassium alginate, magnesium alginate, cellulose, chondroitin sulfate, dermatan sulfate, keratan sulfate, heparin, eparan sulfate, laminin, fibronectin, elastin, polylactic acid, polyglycolic acid, poly(lactic-co-glycolic acid), polycaprolactone, gelatin, albumin, poly(glycolide-co-caprolactone), poly(glycolide-co-trimethylene carbonate), hydroxyapatite, tricalcium phosphate, dicalcium phosphate, demineralised bone matrix, and mixtures thereof.

The composition according to the invention finds advantageous application in the same uses as those listed above for the conjugate.

It should also be understood that all aspects identified as preferable and advantageous for the conjugate procedure should be deemed equally preferable and advantageous also for the conjugates stated above obtainable therefrom, and likewise the compositions and the uses thereof.

It should furthermore be understood that all the possible combinations of the preferred aspects of the invention stated above are deemed to be likewise described and preferred.

Working examples of the present invention are provided below for illustrative and non-limiting purposes.

### EXAMPLES

### Materials

As a substrate, two samples of HA with a molecular weight of 82 kDa and 300 kDa respectively, and an amine derivative of lactose, hereinafter referred to as Lactosamine (Lat-NH₂), were used.

The main reagents used in the HA functionalisation tests and for the synthesis of Lactosamine were:
EDC (N-(3-Dimethylaminopropyl)-N′-ethylcarbodiimide hydrochloride),
NHS (N-Hydroxysuccinimide),
HCTU (O-(1H-6-Chlorobenzotriazole-1-yl)-1,1,3,3-tetramethyluronium hexafluoro-phosphate),
DMTMM (4-(4,6-Dimethoxy-1,3,5-triazin-2-yl)-4-methylmorpholinium chloride),
TEA (Triethylamine),
α-D-Lactose,
NH₃ 28-30% (Ammonium hydroxide solution),
NaCNBH₃ (Sodium cyanoborohydride),
NH₄OAc (Ammonium acetate),
Amberlite® IR-120 (ion exchanger),
SEC Pephadex G-10 exclusion column.

### Example 1

### Lactosamine Synthesis Procedure

360 mg (1 mmol) of α-D-Lactose was solubilised in 20 mL saturated solution of NH₄OAc/EtOH ([c]=0.05M) maintaining the mixture at 40 °C for approximately 30 min. to complete solubilisation. 8 mL 28-30% aqueous ammonia (2.24 g; 131 mmol) was added to the mixture and then 190 mg NaCNBH₃ (3 mmol), and the resulting mixture left under stirring at 90 °C. After 8 hours, the solvent and excess ammonia were evaporated at reduced pressure and the white solid, after being treated several times first with water and then with EtOH alone, was loaded into an Amberlite IR-120(H+) column. (20 eqs.; 15 mL ø=1 h=20 cm) and underwent eluting with deionised water until the acidic conditions disappeared (desalination). The amine product anchored on the sulfonic resin in the first step was recovered through a subsequent elution of 50 mL 10% aqueous ammonia solution and a further 50 mL water until the complete elution of the amine product detected on the TLC plate with 10% sulfuric acid. All the eluted fractions containing the product were evaporated and the residue was treated first with water and subsequently with EtOH until the weight remained constant. 312 mg white product were obtained with yields of 90%.

### Example 2

### Lactosamine Synthesis Procedure

A solution of lactose (3% w/v), benzylamine (5% w/v) and 5-ethyl-2-methylpyridine borane (6% w/v) in water and methanol (3:1) was placed under stirring at a temperature of 55 °C and left to react for 20 hours. Subsequently the mixture was cooled, extracted with dichloromethane and, finally, the aqueous phase evaporated at low pressure, obtaining a crystalline white solid which was then washed with ethyl ether and finally recovered by decantation and dried under reduced pressure. The product was characterised by means of IR and ¹H-NMR spectroscopy. Reaction yield: 90%.

A solution of the derivative thus obtained (4% w/v) in methanol and water (1:1) was placed under magnetic stirring at room temperature. Subsequently, Pd on coal (0.4% w/v) was added and the system thus produced was pressurised with hydrogen. After 48 hours, the system was depressurised, admixed with an equi-volume of water, the solid decanted, and the solution filtered on celite. The solution thus obtained was dried under reduced pressure, providing a white solid. The product thus obtained was characterised by means of IR and ¹H-NMR spectroscopy. Reaction yield: 96%.

### Example 3

### a) Preparation of Hyaluronic Acid Lactosylate

12.02 g (29.83 mmol) sodium hyaluronate was solubilised in 0.7 L deionised water, leaving the mixture under magnetic stirring for 2 h. 4.12 g (14.90 mmol; 0.5 eqs.) condensing agent DMTMM (4-(4,6-Dimethoxy-1,3,5-triazin-2-yl)-4-methylmorpholinium chloride) was added to the homogeneous solution and after 1 h 100 mL aqueous solution containing 5.12 g (14.9 mmol; 0.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. A 25 mL aliquot (1/10; 1.14 g) was lyophilised and subsequently purified according to steps b1, b2, and c, the remaining 225 mL solution (9/10 of the total) was diluted to 350 mL and transferred to a 6-8 kDa membrane and first dialysed for 2 days against 24 litres (12+12 l) 0.1M NaCl saline solution and subsequently 3 days against deionised water. The entire retentate (approximately 1 litre) was then concentrated under reduced pressure to approximately ⅓ of the initial volume and freeze-dried to obtain a white solid.

### b1) Purification by Successive Reprecipitations

570 mg of the sample obtained after a first precipitation and freeze-drying was dissolved in 40 mL deionised water at a concentration of 15 mg/ml and subjected to reprecipitation with 60 mL Isopropanol (4 °C, 60% v/v) enriching the mixture with a few drops of saturated NaCl solution to improve the precipitation step. The suspension was centrifuged at 15000 rpm and the solid obtained was washed several times with isopropanol before being recovered with deionised water and freeze-dried to obtain a white solid.

### b2) Purification by Ion-Exchange Resin Treatment

570 mg of the sample obtained after a first precipitation and freeze-drying was dissolved in 40 mL deionised water ([c]=15 mg/ml) and eluted in an Amberlite cationic column IR-120 (H+) (30 eqs.; 15 mL ø=1 h=20 cm) with a further 60 mL deionised water until the acid solution was completely eluted. The entire eluted mixture was then neutralised with a stoichiometric quantity of NaHCO₃ (106 mg), keeping the mixture under stirring in a vacuum system for approximately 1 h in order to promote acid exchange of the glucuronic acid in the sodium form. The mixture was then concentrated under reduced pressure to approximately ⅓ of the initial volume and freeze-dried to obtain a white solid.

c) Recovery of Lactosamine from the Sample Purified by Ion-Exchange Resin Treatment

From the Amberlite column IR-120 (H+) (30 eqs.; 15 mL ø=1 h=20 cm) used previously, 40 mL 9% NH₃ solution in water was loaded and eluted, followed by a further 80 mL water alone until neutral conditions were obtained. The entire elute was dried under reduced pressure and the residue obtained was treated several times, first with water (5×10 ml) and finally with EtOH, until the weight was constant, obtaining a sample weighing 56 mg.

### Example 4

### a) Preparation of Hyaluronic Acid Lactosylate

12.02 g (29.83 mmol) sodium hyaluronate was solubilised in 0.7 L deionised water, leaving the mixture under magnetic stirring for 2 h. 8.24 g (29.83 mmol; 1.0 eqs.) condensing agent DMTMM (4-(4,6-Dimethoxy-1,3,5-triazin-2-yl)-4-methylmorpholinium chloride) was added to the homogeneous solution and after 1 h 100 mL aqueous solution containing 10.24 g (29.83 mmol; 1.0 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 5

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 15.36 g (44.75 mmol; 1.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 6

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)--4-methylmorpholiniumhloride) was added to the homogeneous solution and after 1 h 100 mL aqueous solution containing 15.36 g (44.75 mmol; 1.5 eqs.) lactosamine was added and the mixture left at pH 6-7 at 25 °C for 24 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 7

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 15.36 g (44.75 mmol; 1.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 8

### a) Preparation of Hyaluronic Acid Lactosylate

12.02 g (29.83 mmol) sodium hyaluronate was solubilised in 0.7 L deionised water, leaving the mixture under magnetic stirring for 2 h. 20.60 g (74.58 mmol; 2.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 25.60 g (74.58 mmol; 2.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 9

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 15.36 g (44.75 mmol; 1.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 10

### a) Preparation of Hyaluronic Acid Lactosylate

12.02 g (29.83 mmol) sodium hyaluronate was solubilised in 0.7 L deionised water, leaving the mixture under magnetic stirring for 2 h. 8.24 g (29.83 mmol; 1.0 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 10.24 g (29.83 mmol; 1.0 eqs.) lactosamine was added and the mixture left at pH 5-8 at 25 °C for 12 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 11

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 15.36 g (44.75 mmol; 1.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 12

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 15.36 g (44.75 mmol; 1.5 eqs.) lactosamine was added and the mixture left at pH 6-7 at 25 °C for 24 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 13

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 20.48 g (59.67 mmol; 1.5 eqs.) lactosamine was added and the mixture left at pH 6-7 at 25 °C for 48 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Example 14

### a) Preparation of Hyaluronic Acid Lactosylate

12.02 g (29.83 mmol) sodium hyaluronate was solubilised in 0.7 L deionised water, leaving the mixture under magnetic stirring for 2 h. 12.36 g (44.75 mmol; 1.5 eqs.) condensing agent DMTMM (4-(4,6-Dimethoxy-1,3,5-triazin-2-yl)-4-methyl-morpholinium chloride) was added to the homogeneous solution and after 1 h 100 mL aqueous solution containing 20.48 g (59.67 mmol; 1.5 eqs.) lactosamine was added and the mixture left at pH 5-8 at 60 °C for 5 h. The product was then isolated by precipitation with 2.5 L isopropanol at 4 °C (75%) enriching the suspension with 3 mL saturated NaCl solution (approximately 1 g salt) to improve the precipitation step and subsequently recovered by centrifugation of the suspension at 15000 rpm for 10 min. The white solid obtained was washed several times with isopropanol, solubilised in 250 mL deionised water. The solution was diluted to 400 mL and transferred to a 6-8 kDa membrane and dialysed first for 2 days against 24 litres (12+12 l) of 0.1M NaCl saline solution and subsequently for 3 days against deionised water. The entire retentate (approximately 1 litre) was then concentrated under reduced pressure to approximately ⅓ of the initial volume and freeze-dried to obtain a white solid.

Alternatively, the white solid obtained after the precipitation and washing step was further purified according to the techniques described in b1 and b2 of Example 3.

### Examples 15-26

The procedures in Examples 3-14 were repeated using 2-chloro-4,6-dimethoxy-1,3,5-triazine (CDMT) instead of DMTMM, likewise obtaining the desired product.

### Example 27

The samples from Examples 3-14 were used to determine the molecular weight (MW) and the degree of substitution (DS).

Molecular weight determination was carried out by size exclusion chromatography (HP-SEC/TDA).

The determination of the degree of substitution was carried out using a two-dimensional hetero-correlated (¹H, ¹³C) HSQC-DEPT nuclear magnetic resonance (NMR) technique following enzymatic hydrolysis of the sample with hyaluronidase.

The results are shown in the table below:

| # | Eq | Yield (%) | MW (Da) | DS % |
|---|---|---|---|---|
| 3 | 1:0.5:0.5 | 84 | 93 | 24 |
| 4 | 1:1:1 | 88 | 115 | 39 |
| 5 | 1:1.5:1.5 | 90 | 95 | 52 |
| 6 | 1:1.5:1.5 | 89 | 93 | 47 |
| 7 | 1:1.5:1.5 | 92 | 254 | 34 |
| 8 | 1:2.5:2.5 | 92 | 278 | 55 |
| 9 | 1:1.5:1.5 | 93 | 525 | 30 |
| 10 | 1:1:1 | 86 | 103 | 33 |
| 11 | 1:1.5:1.5 | 90 | 890 | 28 |
| 12 | 1:1.5:1.5 | 91 | 1560 | 18 |
| 13 | 1:1.5:2.0 | 89 | 157 | 28 |
| 14 | 1:1.5:2.0 | 88 | 86 | 16 |

### Example 28

Samples 14, 13 and 6 were tested on a cell model to evaluate their anti-inflammatory properties.

The anti-inflammatory activity of molecules 14, 13, and 6 was compared with that of the negative control (CTRL, smoker's human bronchial fibroblasts, untreated) and with that of the positive control (TC, smoker's human bronchial fibroblasts incubated with culture of U937 human monocytes activated to macrophages with PMA (50 ug/ml, 24 h) and LPS (1 ug/ml, 1 h) by qPCR analysis of IL-1β gene expression. The following table shows expression of the genes analysed after 4 hours of incubation with compounds 14, 13, and 6:

| | Mean (fold change) | Standard Deviation |
|---|---|---|
| CTRL | 1 | ±0 |
| TC | 58.12 | ±10.18 |
| #14 | 31.85 | ±12.67 |
| #6 | 41.51 | ±9.181 |
| #13 | 34.99 | ±8.040 |

All the compounds demonstrated a significant reduction in IL-1β with respect to the TC.

### Example 29

Samples 14, 13 and 6 were tested on a cell model to evaluate their anti-fibrotic properties. The anti-fibrotic activity of molecules 14, 13, and 6 was compared with that of the negative control (CTRL, human peripheral lung fibroblasts, untreated, NHLF line) and with that of the positive control (TGF-β, human peripheral lung fibroblasts (NHLF) incubated with 5 ng/ml TGF-β for 24 h) by qPCR analysis of COL-1 gene expression. The following table shows expression of the genes analysed after 4 hours of incubation with compounds 14, 13, and 6:

| | Mean (fold change) | Standard Deviation |
|---|---|---|
| CTRL | 1 | ±0 |
| TGF-β | 6.366 | ±0.4154 |
| #13 | 1.524 | ±0.6778 |
| #6 | 2.629 | ±1.408 |
| #14 | 1.289 | ±0.3565 |

All the compounds demonstrated a significant reduction in COL-1 with respect to the TGF-β.

1. Process for conjugating hyaluronic acid, or a pharmaceutically acceptable salt thereof, with a disaccharide or an oligosaccharide, comprising the following steps:
i) providing an aqueous solution of hyaluronic acid or a pharmaceutically acceptable salt thereof,
ii) adding a triazine compound of formula (I), under stirring, to form an ester adduct between the carboxyl group of hyaluronic acid, or a pharmaceutically acceptable salt thereof, and the triazine ring of the compound of formula (I),
iii) waiting at least 15 minutes and adding a primary amine of a disaccharide or oligosaccharide, under stirring, to form a conjugate of hyaluronic acid, or a pharmaceutically acceptable salt thereof, with said disaccharide or oligosaccharide, via amide bond,
iv) promoting precipitation of the conjugate thus obtained, adding a water-miscible organic solvent, thus obtaining a crude conjugate, and possibly washing the precipitated conjugate with further organic solvent possibly mixed with water,
v) separating the crude conjugate, and optionally
vi) purifying the crude conjugate by reprecipitation, by ion-exchange resin treatment, by dialysis, or by the combination and/or repetition thereof, and
vii) obtaining the purified conjugate by drying,
the process being carried out at a temperature of 4-100 °C and at a pH of 5-8,
wherein said triazine compound of formula (I) is: 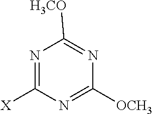
where X a halogen selected from I, Br, Cl and mixtures thereof, or is a group —NR₁R₂ selected from NH₂, NMe₂, NHPr, PhNH, 4-MeC₆H₄NH, 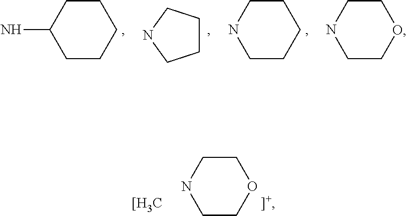
and mixtures thereof.

2. The process of claim 1, wherein said hyaluronic acid of step i) has an average molecular weight of 50-5000 kDa.

3. The process of claim 1, wherein said pharmaceutically acceptable salt of hyaluronic acid of step i) is selected from sodium hyaluronate, potassium hyaluronate, calcium hyaluronate, magnesium hyaluronate, zinc hyaluronate, cobalt hyaluronate, ammonium hyaluronate, tetrabutylammonium hyaluronate and mixtures thereof.

4. The process of claim 1, wherein said primary amine of a disaccharide or an oligosaccharide from trisaccharide to hexasaccharide, is a primary amino derivative of sucrose, maltose, lactose, trehalose, gentiobiose, cellobiose, maltotriose, raffinose, stachyose, maltotetraose, melezitose, isomaltose, panose, nystose, 1-chestose, galactopinitol, galactocyl-lactose, or a mixture thereof.

5. The process of claim 1, wherein, in step iii), a primary amine of a disaccharide is added, which is a primary amino derivative of sucrose, maltose, lactose, trehalose, gentiobiose, cellobiose or a mixture thereof.

6. The process of claim 1, wherein said triazine compound of formula (I) is 4-(4,6-DiMethoxy-1,3,5-Triazin-2-yl)-4-Methyl Morpholinium chloride (DMTMM), 2-chloro-4,6-dimethoxy-1,3,5-triazine (CDMT).

7. The process of claim 1, wherein, when in step vi), the purification of the crude conjugate is by treatment with an ion exchange resin, the following sub-steps are carried out:
vi-a) dissolving the crude conjugate in water to obtain an aqueous solution,
vi-b) treating said aqueous solution with ion exchange resin, using water as eluting agent,
vi-c) isolating the aqueous solution from the ion exchange resin,
vi-d) adjusting the isolated solution to neutral pH.

8. The process of claim 1, wherein, when in step vi), the purification of the crude conjugate is by dialysis, the following sub-steps are carried out:
vi-i) dissolving the crude conjugate in water to obtain an aqueous solution,
vi-ii) dialyzing the solution obtained against aqueous saline solution and/or water through an appropriate membrane.

9. The process of claim 1, wherein, when in step vi), the purification of the crude conjugate is by reprecipitation, the following sub-steps are carried out:
vi-α) dissolving the crude conjugate in water to obtain an aqueous solution,
vi-β) adding an organic solvent miscible in water, equal to or different from that of step iv), thus promoting the reprecipitation of the conjugate,
vi-γ) washing the precipitated conjugate with further organic solvent, thus obtaining the purified conjugate.

10. The process of claim 1, said process being carried out at a temperature of 15-75 °C and a pH of 6-7.

11. The process of claim 1, wherein hyaluronic acid or a pharmaceutically acceptable salt thereof, and triazine compound of formula (I), in step ii), are in molar ratio of 3:1 to 1:3.

12. The process of claim 11, wherein hyaluronic acid or a pharmaceutically acceptable salt thereof, and said a primary amine, in step iii), are in molar ratio of 3:1 to 1:3.

13. Conjugate of hyaluronic acid, or a pharmaceutically acceptable salt thereof, and a disaccharide or an oligosaccharide, having 10-50%, of average substitution degree of the carboxylic group with the amide bond to said disaccharide or oligosaccharide, and being obtained by the process of claim 11.

14. The conjugate of claim 13 in form of a composition comprising at least one pharmacologically active substance and/or at least one substance optionally having a biological function, wherein:
said pharmacologically active substance is selected from antibiotics, anti-infectives, antimicrobials, antivirals, cytostatic, cytotoxic, antitumor, anti-inflammatory, healing, anesthetic, analgesic, vasoconstrictor, agonists and antagonists, cholinergic or adrenergic, antithrombotic, anticoagulant, haemostatic and their fragments, peptides, polynucleotides, growth factors, enzymes, vaccines, and combinations thereof, and
said substance optionally having a biological function is chosen from collagen, fibrinogen, fibrin, alginic acid, sodium alginate, potassium alginate, magnesium alginate, cellulose, chondroitin sulfate, dermatan sulfate, keratan sulfate, heparin, heparan sulfate, laminin, fibronectin, elastin, polylactic acid, polyglycolic acid, poly (lactic-co-glycolic acid), polycaprolactone, gelatin, albumin, poly (glycolide-co-caprolactone), poly (glycolide-co-trimethylene carbonate), hydroxyapatite, tricalciophosphate, dicalciophosphate, bone matrix demineralized, and mixtures thereof.

15. The conjugate of claim 13, in the form of powder, solution, emulsion, gel, ointment, drops, aerosol, spray, or in an injectable form.

16. The conjugate of claim 13 in the form of
a biomaterial or scaffold for cell growth.

17. The process of claim 5, wherein, in step iii), a primary amine of a disaccharide is added, which is a primary amino derivative of lactose.

18. The process of claim 6, wherein said triazine compound of formula (I) is 4-(4,6-DiMethoxy-1,3,5-Triazin-2-yl)-4-Methyl Morpholinium chloride (DMTMM).

19. A method of treating a disease ascribable to an altered expression of galectins in a patient in need thereof, the method comprising:
administering to said patient a therapeutically effective amount of the conjugate of claim 13,
wherein the disease includes non-alcoholic steatohepatitis, plaque psoriasis, rheumatoid arthritis, osteoarthritis, neoplasms, adhesions, and dermal, pulmonary, renal, and cardiovascular fibrotic processes.
